(19) United States
(12) Patent Application Publication
(10) Pub. No.: US 2019/0241485 A1
(43) Pub. Date: Aug. 8, 2019

(54) Catalysts for Producing Paraxylene by Methylation of Benzene and/or Toluene

(71) Applicant: ExxonMobil Chemical Patents Inc., Baytown, TX (US)

(72) Inventors: Wenyih F. Lai, Bridgewater, NJ (US); Tan-Jen Chen, Seattle, WA (US); Seth M. Washburn, Houston, TX (US)

(21) Appl. No.: 16/216,789

(22) Filed: Dec. 11, 2018

Related U.S. Application Data
(60) Provisional application No. 62/609,458, filed on Dec. 22, 2017.

(51) Int. Cl. C07C 2/66; B01J 29/06; B01J 29/70; B01J 37/00

(52) U.S. Cl. CPC C07C 2/66; B01J 29/061; C07C 15/08; B01J 37/0027; B01J 29/7038

(57) ABSTRACT

Embodiments disclosed herein include a process for producing paraxylene and catalyst for use in processes for producing paraxylene. In an embodiment, the process includes contacting an aromatic hydrocarbon feed comprising benzene and/or toluene with an alkylating reagent comprising methanol and/or dimethyl ether in at least one alkylation reaction zone in the presence of an alkylation catalyst comprising a molecular sieve having a Constrain Index less than 5 and under alkylation conditions. The alkylation catalyst comprises at least one of a rare earth metal or alkaline earth metal and a binder, and a majority of the at least one rare earth metal or alkaline earth metal is deposited on the molecular sieve. In addition, the process includes producing an alkylated aromatic product comprising xylenes.

### CROSS-REFERENCE OF RELATED APPLICATIONS

This application claims priority to and the benefit of U.S. Ser. No. 62/609,458, filed Dec. 22, 2017 and is incorporated by reference in its entirety.

### TECHNICAL FIELD

This disclosure generally relates to a catalysts for the methylation of benzene and/or toluene to produce xylenes, particularly paraxylene.

### BACKGROUND

Xylenes are valuable precursors in the chemical industry. Of the three xylene isomers, paraxylene is the most important since it is a starting material for manufacturing terephthalic acid, which is itself a valuable intermediate in the production of synthetic polyester fibers, films, and resins. Currently, the demand for paraxylene is growing at an annual rate of 5-7%.

One known route for the manufacture of paraxylene is by the methylation of benzene and/or toluene. For example, U.S. Pat. No. 6,504,072 discloses a process for the selective production of paraxylene which comprises reacting toluene with methanol under alkylation conditions in the presence of a catalyst comprising a porous crystalline material having a Diffusion Parameter for 2,2 dimethylbutane of about 0.1-15 sec⁻¹ when measured at a temperature of 120° C. and a 2,2 dimethylbutane pressure of 60 torr (8 kPa). The porous crystalline material is preferably a medium-pore zeolite, particularly ZSM-5, which has been severely steamed at a temperature of at least 950° C. The alkylation conditions include a temperature between about 500 and 700° C., a pressure of between about 1 atmosphere and 1000 psig (100 and 7000 kPa), a weight hourly space velocity between about 0.5 and about 1000 and a molar ratio of toluene to methanol of at least about 0.2.

In addition, U.S. Pat. No. 6,642,426 discloses a process for alkylating an aromatic hydrocarbon reactant, especially toluene, with an alkylating reagent comprising methanol to produce an alkylated aromatic product, comprising: introducing the aromatic hydrocarbon reactant into a reactor system at a first location, wherein the reactor system includes a fluidized bed reaction zone comprising a temperature of 500 to 700° C. and an operating bed density of about 300 to 600 kg/m³, for producing the alkylated aromatic product; introducing a plurality of streams of said alkylating reactant directly into said fluidized bed reaction zone at positions spaced apart in the direction of flow of the aromatic hydrocarbon reactant, at least one of said streams being introduced at a second location downstream from the first location; and recovering the alkylate aromatic product, produced by reaction of the aromatic reactant and the alkylating reagent, from the reactor system. The preferred catalyst is ZSM-5 which has been selectivated by high temperature steaming

As exemplified by the U.S. Patents discussed above, current processes for the alkylation of benzene and/or toluene with methanol are conducted at high temperatures, i.e., between 500 to 700° C. in the presence of a medium pore size zeolite, particularly ZSM-5. This results in a number of problems, particularly in that catalyst life per cycle is relatively short and so frequent regeneration of the catalyst is required. In addition, the existing processes typically result in significant quantities of methanol being converted to ethylene and other light olefins which reduces the yield of desirable products, such as xylenes, and increases recovery costs.

There is therefore a need for an improved process and/or catalysts for the alkylation of benzene and/or toluene with methanol (or dimethyl ether), which increases the paraxylene selectivity of the catalyst and produces a higher than equilibrium amount of paraxylene.

### BRIEF SUMMARY

Some embodiments disclosed herein are directed to a process for producing paraxylene. In an embodiment, the process includes contacting an aromatic hydrocarbon feed comprising benzene and/or toluene with an alkylating reagent comprising methanol and/or dimethyl ether in at least one alkylation reaction zone in the presence of an alkylation catalyst comprising a molecular sieve having a Constrain Index less than 5 and under alkylation conditions. The alkylation catalyst comprises at least one of a rare earth metal or alkaline earth metal and a binder, and a majority of the at least one rare earth metal or alkaline earth metal is deposited on the molecular sieve. In addition, the process includes producing an alkylated aromatic product comprising xylenes.

Other embodiments disclosed herein are directed to a process for producing paraxylene. In an embodiment, the process includes contacting an aromatic hydrocarbon feed comprising benzene and/or toluene with an alkylating reagent comprising methanol and/or dimethyl ether in at least one alkylation reaction zone in the presence of an alkylation catalyst comprising a molecular sieve of the MWW framework structure under alkylation conditions. The alkylation catalyst comprises lanthanum and a binder, and wherein a majority of the lanthanum is deposited on the molecular sieve. In addition, the process includes producing an alkylated aromatic product comprising xylenes.

### BRIEF DESCRIPTION OF THE FIGURES

FIG. 1 is a schematic side view of a mulling operation for forming a catalyst in accordance with at least some embodiments disclosed herein.

FIG. 2 is a schematic top view of the mulling operation of FIG. 1.

FIGS. 3-6 are plots showing the comparative performance data for an La modified MCM-49 catalyst and an unmodified MCM-49 catalyst.

### DETAILED DESCRIPTION

The following discussion is directed to various embodiments. However, it should be appreciated that the embodiments disclosed herein have broad application, and that the discussion of any embodiment is meant only to be exemplary of that embodiment, and not intended to suggest that the scope of the disclosure, including the claims, is limited to that embodiment. In the drawings, certain features and components herein may be shown exaggerated in scale or in somewhat schematic form and some details of conventional elements may not be shown in interest of clarity and conciseness. All documents described herein are incorporated by reference, including any priority documents and/or testing procedures, to the extent they are not inconsistent with this text. When numerical lower limits and numerical upper limits are listed herein, ranges from any lower limit to any upper limit are contemplated.

As used herein, the term “Cn” hydrocarbon wherein n is a positive integer, e.g., 1, 2, 3, 4, 5, etc., means a hydrocarbon having n number of carbon atom(s) per molecule. The term “Cn+” hydrocarbon wherein n is a positive integer, e.g., 1, 2, 3, 4, 5, etc., means a hydrocarbon having at least n number of carbon atom(s) per molecule. The term “Cn-” hydrocarbon wherein n is a positive integer, e.g., 1, 2, 3, 4, 5, etc., as used herein, means a hydrocarbon having no more than n number of carbon atom(s) per molecule.

As used herein, the terms “alkylating” and “methylating”, or “alkylation” and “methylation” may be used interchangeably.

Constraint Index is a convenient measure of the extent to which a molecular sieve provides control of molecules of varying sizes to its internal structure. The method by which Constraint Index is determined is described fully in U.S. Pat. No. 4,016,218, to which reference is made for details of the method.

Embodiments disclosed herein provide catalysts for use in an alkylation process for producing xylenes, particularly paraxylene, and alkylation processes utilizing such catalysts. In some embodiments, the catalyst disclosed herein can be utilized in alkylation processes under relatively mild conditions to produce xylenes with less light gas by-products and longer catalyst cycle life than conventional high temperature processes. In the catalysts of at least some of the embodiments disclosed herein, a zeolite of the MWW framework type is modified with a rare earth metal and/or alkaline earth metal to improve selectivation toward xylenes, particularly paraxylene. In the alkylation process, an aromatic hydrocarbon feed comprising benzene and/or toluene is contacted with an alkylating reagent comprising methanol and/or dimethyl ether in at least one alkylation reaction zone in the presence of the alkylation catalyst under alkylation conditions.

In at least some embodiments, the process is effective to convert the benzene and/or toluene to xylenes with essentially 100% methanol conversion and substantially no light gas make. The high methanol utilization is surprising in light of the methanol utilization in the prior art toluene and/or benzene methylation processes, and results in the substantial advantages of less coke formation, which increases the catalyst life. Furthermore, in conventional processes, it is preferred to co-feed steam into the reactor with the methanol to minimize the methanol side reactions, and the steam negatively impacts catalyst life. With the nearly 100% of the methanol reacting with aromatic rings to produce aromatics in the processes disclosed herein, there is no need to co-feed steam, decreasing the energy demands of the process and increasing catalyst life.

The methanol selectivity to xylenes in the processes disclosed herein is typically on the order of 80%, with the main by-products being benzene and C9+ aromatics. The benzene can be separated from the alkylation effluent and recycled back to the alkylation reaction zone(s), while the C9+ aromatics can be separated for blending into the gasoline pool or transalkylated with additional benzene and/or toluene to make additional xylenes. Moreover, the use of a larger pore molecular sieve minimizes diffusion limitations and allows the alkylation to be carried out at commercially viable WHSVs. Additionally, when a toluene feed (one having at least 90 wt % of toluene) is used, more alkylating agent reacts with the toluene, versus other molecules such as alkylating agent or by-products of the reaction, to produce xylenes as compared to existing processes.

The amount of paraxylene in the xylenes product can be increased up to at least 35 wt % by selectivating the alkylation catalyst. In one embodiment, the alkylation catalyst is selectivated ex-situ by modifying the catalyst with a rare earth metal and/or alkaline earth metal. For example, in some embodiments, the alkylation catalyst may be modified with lanthanum (La) and/or strontium (Sr), prior to utilizing the alkylation catalyst in the alkylation process.

Molecular sieves for use in embodiments disclosed herein may include those having a Constraint Index less than 5. Suitable examples of such molecular sieves include, for example, zeolite beta, zeolite Y, Ultrastable Y (USY), Ultrahydrophobic Y (UHP-Y), Dealuminized Y (Deal Y), mordenite, ZSM-3, ZSM-4, ZSM-12, ZSM-14, ZSM-18, ZSM-20 and mixtures thereof. Zeolite ZSM-3 is described in U.S. Pat. No. 3,415,736. Zeolite ZSM-4 is described in U.S. Pat. No. 4,021,947. Zeolite ZSM-12 is described in U.S. Pat. No. 3,832,449. Zeolite ZSM-14 is described in U.S. Pat. No. 3,923,636. Zeolite ZSM-18 is described in U.S. Pat. No. 3,950,496. Zeolite ZSM-20 is described in U.S. Pat. No. 3,972,983. Zeolite Beta is described in U.S. Pat. Nos. 3,308,069, and Re. No. 28,341. Low sodium Ultrastable Y molecular sieve (USY) is described in U.S. Pat. Nos. 3,293,192 and 3,449,070. Ultrahydrophobic Y (UHP-Y) is described in U.S. Pat. No. 4,401,556. Dealuminized Y zeolite (Deal Y) may be prepared by the method found in U.S. Pat. No. 3,442,795. Zeolite Y and mordenite are naturally occurring materials but are also available in synthetic forms, such as TEA-mordenite (i.e., synthetic mordenite prepared from a reaction mixture comprising a tetraethylammonium directing agent). TEA-mordenite is disclosed in U.S. Pat. Nos. 3,766,093 and 3,894,104.

One preferred class of molecular sieve suitable for use in the embodiments disclosed herein, and having a Constraint Index less than 5, are crystalline microporous materials of the MWW framework type. As used herein, the term “crystalline microporous material of the MWW framework type” includes one or more of:
molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, the entire content of which is incorporated as reference);
molecular sieves made from a common second degree building block, being a 2-dimensional tiling of such MWW framework topology unit cells, forming a monolayer of one unit cell thickness, preferably one c-unit cell thickness;
molecular sieves made from common second degree building blocks, being layers of one or more than one unit cell thickness, wherein the layer of more than one unit cell thickness is made from stacking, packing, or binding at least two monolayers of MWW framework topology unit cells. The stacking of such second degree building blocks can be in a regular fashion, an irregular fashion, a random fashion, or any combination thereof; and
molecular sieves made by any regular or random 2-dimensional or 3-dimensional combination of unit cells having the MWW framework topology.

Crystalline microporous materials of the MWW framework type include those molecular sieves having an X-ray diffraction pattern including d-spacing maxima at 12.4±0.25, 6.9±0.15, 3.57±0.07 and 3.42±0.07 Angstrom. The X-ray diffraction data used to characterize the material are obtained by standard techniques using the K-alpha doublet of copper as incident radiation and a diffractometer equipped with a scintillation counter and associated computer as the collection system.

Examples of crystalline microporous materials of the MWW framework type include MCM-22 (described in U.S. Pat. No. 4,954,325), PSH-3 (described in U.S. Pat. No. 4,439,409), SSZ-25 (described in U.S. Pat. No. 4,826,667), ERB-1 (described in European Patent No. 0293032), ITQ-1 (described in U.S. Pat. No. 6,077,498), ITQ-2 (described in International Publication No. W097/17290), MCM-36 (described in U.S. Pat. No. 5,250,277), MCM-49 (described in U.S. Pat. No. 5,236,575), MCM-56 (described in U.S. Pat. No. 5,362,697), UZM-8 (described in U.S. Pat. No. 6,756,030), UZM-8HS (described in U.S. Pat. No. 7,713,513), UZM-37 (described in U.S. Pat. No. 7,982,084), EMM-10 (described in U.S. Pat. No. 7,842,277), EMM-12 (described in U.S. Pat. No. 8,704,025), EMM-13 (described in U.S. Pat. No. 8,704,023), MIT-1 (described by Luo et. al, in Chemical Science, 2015, Vol. 6, pp. 6320-6324) and mixtures thereof, with MCM-49 generally being preferred.

In some embodiments, the crystalline microporous material of the MWW framework type employed in the embodiments disclosed herein may be contaminated with other crystalline materials, such as ferrierite or quartz. These contaminants may be present in quantities ≤10% by weight, normally ≤5% by weight.

Additionally or alternatively, the molecular sieves useful in the embodiments disclosed herein may be characterized by a molar ratio of silicon to aluminum (i.e., a Si/Al ratio). In particular embodiments, the molecular sieves suitable herein include those having a Si/Al ratio of less than 100, preferably about 15 to 50.

The molecular sieve catalyst may be selectivated to produce a higher than equilibrium amount of paraxylene (i.e., more than about 23 wt % of paraxylene, based on the total amount of xylenes) in the product mixture. In one embodiment, the concentration of paraxylene in the xylene fraction is at least 35 wt %, preferably at least 40 wt %, and more preferably at least 45 wt %. The selectivation of the catalyst may be conducted ex-situ by modifying the molecular sieve catalyst with a rare earth metal and/or alkaline earth metal. As used herein, the target paraxylene selectivity means at least 35 wt %, preferably at least 40 wt %, and more preferably at least 45 wt % of paraxylene in the xylenes fraction.

In particular, in the embodiments disclosed herein, the molecular sieve may be combined with at least one modifier (e.g., an oxide modifier), such as at least one oxide selected from at least one of a rare earth metal and an alkaline earth metal. Most preferably, said at least one oxide modifier is selected from oxides of lanthanum and strontium. In some cases, the molecular sieve may be combined with more than one oxide modifier. For example, in some embodiments, the molecular sieve may be combined with oxides of one or more of boron, magnesium, calcium, and phosphorus in addition to the oxides of the rare earth and/or alkaline earth metals described above.

In some embodiments, the total amount of rare earth and/or alkaline earth metal present in the catalyst, as measured on an elemental basis, may be between about 1 and about 10 wt %, and preferably is between about 1 and about 5 wt %, based on the weight of the final catalyst.

Modification of the molecular sieve with a rare earth and/or alkaline earth metal may be accomplished by direct synthesis, such has by contacting the molecular sieve material, either alone or in combination with a binder or matrix material, with a solution of an appropriate rare earth and/or alkaline earth metal containing compound. In some embodiment, the rare earth and/or alkaline earth metal is combined with the molecular sieve via impregnation. Where the modifier includes phosphorus, incorporation of modifier into the catalyst is conveniently achieved by the methods described in U.S. Pat. Nos. 4,356,338, 5,110,776, 5,231,064, and 5,348,643, the entire disclosures of which are incorporated herein by reference.

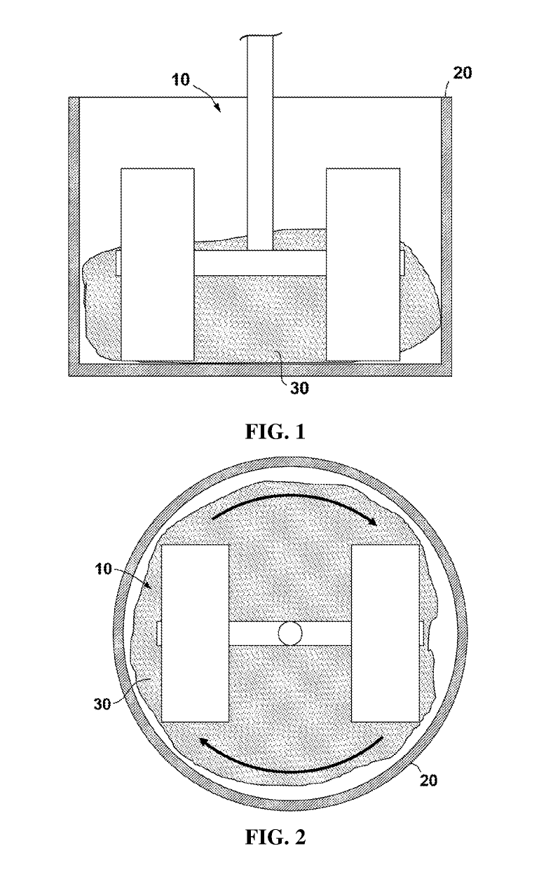

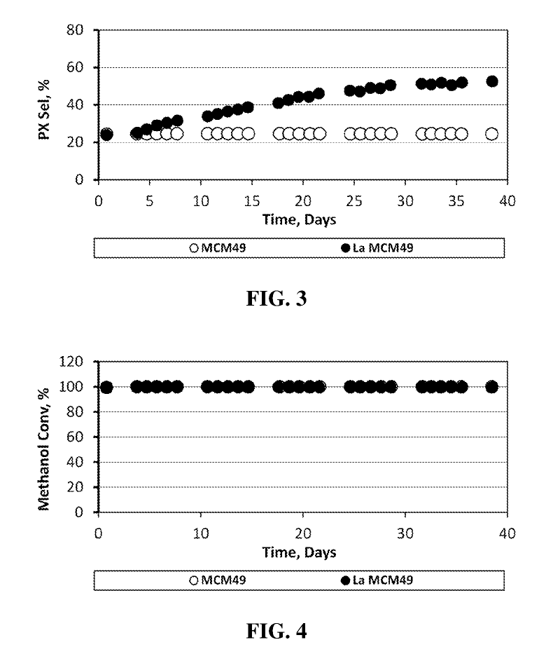

Referring to FIGS. 1 and 2, in still other embodiments, the rare earth and/or alkaline earth metal may be combined with the molecular sieve via muller addition or a mulling operation. In particular, in these embodiments an oxide of a rare earth or alkaline earth metal is added to an extrusion mixture 30, and the combined materials are subject to a mulling operation. In one example of such a mulling operation, the mixture 30 is placed in a container or vessel 20 and is subject to direct high pressure (e.g., via a roller(s) or other mechanical device(s) 10) at relatively low temperatures (e.g., room temperatures) to facilitate mixing and combination of the ingredients. Depending on the composition of the mixture 30, the mulling operation can be used to achieve a desired positioning of the metal oxide within the catalyst.

Specifically, in at least some embodiments, it is desirable to deposit the rare earth and/or alkaline earth metal directly on the molecular sieve itself (or at least mostly on the molecular sieve), as opposed to more or less even distribution on the molecular sieve and the binder. For example, in some embodiments more than 50% (e.g., at least 60, 70, 80, 90, 99%) of the rare earth and/or alkaline earth metal of the catalyst is deposited on the molecular sieve. In still others of these embodiments, substantially all of the rare earth and/or alkaline earth metal of the catalyst is deposited on the molecular sieve rather than the binder. During manufacturing of these embodiments utilizing a mulling operation, the rare earth and/or alkaline earth metal (or precursor thereof) is mulled together first with the molecular sieve (e.g., the crystals of the molecular sieve itself) to facilitate combination of the two compositions and therefore deposition of the metal onto the molecular sieve. Thereafter, the combined metal and molecular sieve is again mulled with other catalyst ingredients (e.g., the binder) to facilitate formation of the final extrudable catalyst mixture.

The catalyst may additionally be selectivated, either before introduction into the aromatization reactor or in-situ in the reactor, by contacting the catalyst with a selectivating agent, such as silicon, silica, silicalite, steam, coke, or a combination thereof. In one embodiment, the catalyst is silica-selectivated by contacting the catalyst with at least one organosilicon in a liquid carrier and subsequently calcining the silicon-containing catalyst in an oxygen-containing atmosphere, e.g., air, at a temperature of 350 to 550° C. A suitable silica-selectivation procedure is described in U.S. Pat. No. 5,476,823, the entire contents of which are incorporated herein by reference. In another embodiment, the catalyst is selectivated by contacting the catalyst with steam. Steaming of the zeolite is effected at a temperature of at least about 900° C., preferably about 950 to about 1075° C., and most preferably about 1000 to about 1050° C., for about 10 minutes to about 10 hours, preferably from 30 minutes to 5 hours. The selectivation procedure, which may be repeated multiple times, alters the diffusion characteristics of the molecular sieve and may increase the xylene yield.

In addition to, or in place of, silica or steam selectivation, the catalyst may be subjected to coke selectivation. This optional coke selectivation typically involves contacting the catalyst with a thermally decomposable organic compound at an elevated temperature in excess of the decomposition temperature of said compound but below the temperature at which the crystallinity of the molecular sieve is adversely affected. Further details regarding coke selectivation techniques are provided in the U.S. Pat. No. 4,117,026, incorporated by reference herein. In some embodiments, a combination of silica selectivation and coke selectivation may be employed.

The above molecular sieves may be used as the alkylation catalyst employed herein without any binder or matrix. Alternatively, the molecular sieves may be composited with another material which is resistant to the temperatures and other conditions employed in the alkylation reaction. Such materials include active and inactive materials and synthetic or naturally occurring zeolites as well as inorganic materials such as clays and/or oxides such as alumina, silica, silica-alumina, zirconia, titania, magnesia or mixtures of these and other oxides. The latter may be either naturally occurring or in the form of gelatinous precipitates or gels including mixtures of silica and metal oxides. Clays may also be included with the oxide type binders to modify the mechanical properties of the catalyst or to assist in its manufacture. Use of a material in conjunction with the molecular sieve, i.e., combined therewith or present during its synthesis, which itself is catalytically active may change the conversion and/or selectivity of the catalyst. Inactive materials suitably serve as diluents to control the amount of conversion so that products may be obtained economically and orderly without employing other means for controlling the rate of reaction. These materials may be incorporated into naturally occurring clays, e.g., bentonite and kaolin, to improve the crush strength of the catalyst under commercial operating conditions and function as binders or matrices for the catalyst. The relative proportions of molecular sieve and inorganic oxide matrix vary widely, with the sieve content ranging from about 1 to about 90 wt % and more usually, particularly, when the composite is prepared in the form of beads, in the range of about 2 to about 80 wt % of the composite.

Additionally, the catalysts disclosed herein may be referred to in reference to their “alpha value.” The alpha value is a measure of the cracking activity of a catalyst and is described in U.S. Pat. No. 3,354,078 and in the Journal of Catalysis, Vol. 4, p. 527 (1965); Vol. 6, p. 278 (1966) and Vol. 61, p. 395 (1980), each incorporated herein by reference. The experimental conditions of the test used herein included a constant temperature of 538° C. and a variable flow rate as described in detail in the Journal of Catalysis, Vol. 61, p. 395 (1980). In some embodiments, the alpha value of the catalyst disclosed herein is 150 or higher, such as 250 or higher, or between 250 and 700.

The feeds to the present process comprise an aromatic hydrocarbon feed, comprising benzene and/or toluene, and an alkylating reagent comprising methanol and/or dimethyl ether. Any refinery aromatic feed can be used as the source of the benzene and/or toluene, although in some embodiments it may be desirable to use an aromatic hydrocarbon feed which comprises at least 90 wt % toluene. In addition, in some embodiments, it may be desirable to pre-treat the aromatic hydrocarbon feed to remove catalyst poisons, such as nitrogen and sulfur-compounds. In other embodiments, the feed may further include non-aromatics, such as a refinery aromatic feed from which the non-aromatics have not been extracted.

The alkylation process of the embodiments disclosed herein may be generally conducted at a temperature between about 500° C. and about 700° C., preferably between about 550 and 650° C. Operating pressures will vary with temperature but generally are at least 700 kPa-a, such as at least 1000 kPa-a, for example at least 1500 kPa-a, or at least 2000 kPa-a, up to about 7000 kPa-a, for example up to about 6000 kPa-a, up to about 5000 kPa-a. In terms of ranges, operating pressures may range from 700 kPa-a to 7000 kPa-a, for example from 1000 kPa-a to 6000 kPa-a, such as from 2000 kPa-a to 5000 kPa-a. Suitable weight hourly space velocity (WHSV) values based on total aromatic and alkylating reagent feeds are in the range from 50 to 0.5 hr⁻¹, such as in the range from 10 to 1 hr⁻¹. In some embodiments, at least part of the aromatic feed, the methanol alkylating reagent and/or the alkylation effluent may be present in the alkylation reaction zone in the liquid phase.

In some embodiments, the present alkylation process may be conducted at relatively low temperatures, namely less than 500° C., such as less than 475° C., or less than 450° C., or less than 425° C., or less than 400° C. In order to provide commercially viable reaction rates, the process may be conducted at temperatures of at least 250° C., such as least 275° C., for example least 300° C. In terms of ranges, the process in these embodiments may be conducted at temperatures ranging from 250 to less than 500° C., such as from 275 to 475° C., for example from 300 to 450° C. In embodiments where a lower operating temperature is used (e.g., a temperature generally less than 500° C.), the life of the alkylation catalyst may be enhanced as compared with higher temperature processes since methanol decomposition is much less at the lower reaction temperature.

The alkylation reaction can be conducted in any known reactor system including, but not limited to, a fixed bed reactor, a moving bed reactor, a fluidized bed reactor and a reactive distillation unit. In addition, the reactor may comprise a single reaction zone or multiple reaction zones located in the same or different reaction vessels. In addition, injection of the methanol/dimethyl ether alkylating agent can be effected at a single point in the reactor or at multiple points spaced along the reactor.

The product of the alkylation reaction comprises xylenes, benzene and/or toluene (both residual and coproduced in the process), C₉₊ aromatic hydrocarbons, co-produced water, oxygenate by-products, and in some cases, unreacted methanol. It is, however, generally preferred to operate the process so that all the methanol is reacted with the aromatic hydrocarbon feed and the alkylation product is generally free of residual methanol. The alkylation product is also generally free of light gases generated by methanol decomposition to ethylene and other olefins. In some embodiments, the organic component of the alkylation product may contain at least 80 wt % xylenes and paraxylene may make up at least 35 wt % of the xylene fraction.

After separation of the water, the alkylation product may be fed to a separation section, such as one or more distillation columns, to recover the xylenes and separate the benzene and toluene from the C₉₊ aromatic hydrocarbon by-products. The resulting benzene and/or toluene may be recycled to the alkylation reaction zone, while C₉₊ aromatics can be recovered for blending into the gasoline pool or transalkylated with additional benzene and/or toluene to make additional xylenes. Oxygenate by-products may be removed from the alkylation product by any means known in the art, such as adsorption as described in U.S. Pat. Nos. 9,012,711, 9,434,661, and 9,205,401; caustic wash as described in U.S. Pat. No. 9,294,962; crystallization as disclosed in U.S. Pat. Nos. 8,252,967, 8,507,744, and 8,981,171; and conversion to ketones as described in U.S. Patent Publication Nos. 2016/0115094 and 2016/0115103.

The xylenes recovered from the alkylation product and any downstream C₉₊ transalkylation process may be sent to a paraxylene production loop. The latter comprises paraxylene separation section, where paraxylene is conventionally separated by adsorption or crystallization, or a combination of both, and recovered. When paraxylene is separated by adsorption, the adsorbent used preferably contains a zeolite. Typical adsorbents used include crystalline alumino-silicate zeolites either natural or synthetic, such as for example zeolite X, or Y, or mixtures thereof. These zeolites are preferably exchanged by cations such as alkali or alkaline earth or rare earth cations. The adsorption column is preferably a simulated moving bed column (SMB) and a desorbent, such as for example paradiethylbenzene, paradifluorobenzene, diethylbenzene, or toluene, or mixtures thereof, is used to recover the selectively adsorbed paraxylene. Commercial SMB units that are suitable for use in the inventive process are PAREX™ or ELUXYL™.

Particular reference will now be made to the following non-limiting example.

### EXAMPLE 1

La-containing MCM-22 crystals were synthesized from a mixture prepared from 990 g of water, 80 g of Hexamethylethleneimine (HMI) (99% solution), 275 g of silica, 74 g of sodium aluminate solution (45%), and 13.5 g of 50% sodium hydroxide solution, and 15.2 g of lanthanum nitrate hexahydrate in 50 g of deionized water. The mixture had the molar compositions shown in Table 1 below.

TABLE 1

| | |
|---|---|
| SiO₂/Al₂O₃ | ~22.5 |
| H₂O/SiO₂ | ~14 |
| OH⁻/SiO₂ | ~0.15 |
| Na⁺/SiO₂ | ~0.215 |
| HMI/SiO₂ | ~0.19 |

The mixture was reacted at 320° F. (160° C.) in a 52-liter autoclave with stirring at 250 RPM for 72 hours. The product was filtered, washed with deionized water and dried at 250° F. (121° C.). The x-ray diffraction pattern of the as-synthesized material showed the typical pure phase of MCM-22 topology. A scanning electron microscope (SEM) image of the as-synthesized material showed typical morphology of layered crystals. The resulting as-synthesized La-MCM-22 crystals showed a SiO₂/Al₂O₃ molar ratio of about 21.1 and 1.74 wt % of La.

The as-synthesized crystals La-MCM-22 crystals were converted into the hydrogen form by three ion exchanges with ammonium nitrate solution at room temperature, followed by drying at 250° F. (121° C.) and calcination at 1000° F. (538° C.) for 6 hours. The resulting H-form, La-MCM-22 crystals had a total (micro+meso) surface area of 582 (515+67) m²/g, hexane sorption of 99.4 mg/g, and the alpha value of 770.

### EXAMPLE 2

A catalyst was made from a mixture of 80 parts (basis: calcined 538° C.) of the La-MCM-22 crystals from Example 1 and 20 parts of Versal™ 300 pseudoboehmite alumina (basis: calcined 538° C.) that was combined in a mulling operation. Sufficient water was added to produce an extrudable paste. The mixture of La-MCM-22 crystals, pseudoboehmite alumina, and water was then extruded into a 80/20 1/20″ Q extrudate, and then dried at 121° C. The dried extrudate was calcined in nitrogen (Na) at 538° C. to decompose and remove the organic template. The N₂-calcined extrudate was humidified with saturated air and exchanged with 1 N ammonium nitrate to remove sodium. After ammonium nitrate exchange, the extrudate was washed with deionized water to remove residual nitrate ions prior to drying. The ammonium-exchanged extrudate was dried at 116° C. and calcined in air at 534° C. The H-formed extrudate had an Alpha of 610, Hexane sorption of 93.9 and surface area of 518 m²/g.

### EXAMPLE 3

An unmodified MCM-49 catalyst was made from a mixture of 80 parts (basis: calcined 538° C.) of MCM-49 crystals and 20 parts high surface area Versal™ 300 alumina (basis: calcined 538° C.) that was combined in a mulling operation. The mixture of MCM-49, Versal™ 300 alumina, and water was extruded into 1/20″ Quadra-lobes and then dried in a hot pack oven at 121° C. overnight. The dried extrudate was calcined in nitrogen (N₂) at 538° C. to decompose and remove the organic template. The N₂-calcined extrudate was humidified with saturated air and exchanged with 1 N ammonium nitrate to remove sodium. After ammonium nitrate exchange, the extrudate was washed with deionized water to remove residual nitrate ions prior to drying. The ammonium-exchanged extrudate was dried at 121° C. overnight and calcined in air at 538° C. The H-formed extrudate had an Alpha of 520, Hexane sorption of ˜91, a total (micro+meso) surface area of 536/(353+184), and a collidine adsorption of ˜71 umoles/g.

### EXAMPLE 4

An LaOx modified MCM-49 catalyst (i.e., an La/MCM-49 catalyst) was prepared by impregnation of a Lanthanum Nitrate Pentahydrate solution on the catalyst of Example 3. The mixture was then dried and calcined at 1000° F. (538° C.) for 3 hrs. The modified extrudate had an La content of 3.45 wt %, an Alpha value of 350 and a total surface area of 450 m²/g. Collidine adsorption was reduced to 62.6 umoles/g after modification.

### EXAMPLE 5

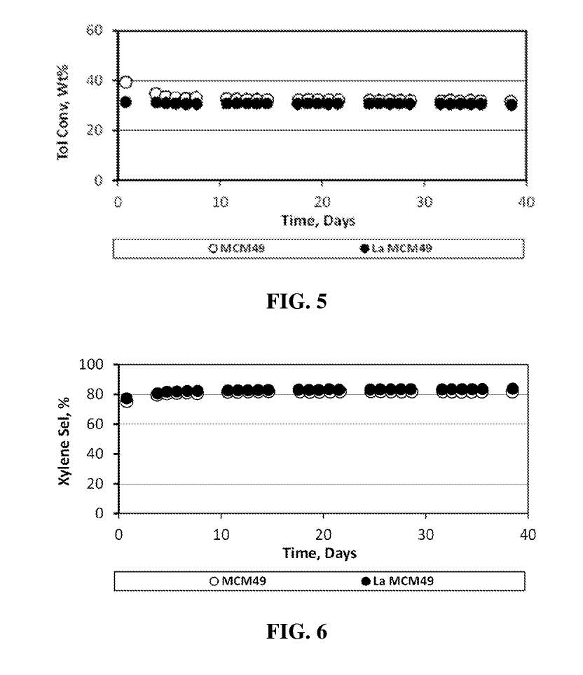

The La/MCM-49 catalyst of Example 4 was subjected to a feed comprising a molar ratio of Methanol to Toluene or 1:3 at a WHSV of 3.5 hr⁻¹ a temperature of about 350° C., and a pressure of 500-600 psig. In addition, the unmodified MCM-49 catalyst of Example 3 was prepared and also subjected to the same feed and conditions as described above. FIGS. 3-6 show the performance comparison of the La/MCM-49 and unmodified MCM-49 catalyst.

In particular, as is shown in FIG. 3, para-xylene selectivity (i.e., the concentration of para-xylene in the xylene product from the reaction) of the La/MCM-49 catalyst was shown to be 24% initially but then increased to approximately 50% over the first thirty days on stream. By contrast the unmodified MCM-49 showed PX selectivity of 24-25% throughout the entire time on stream. It should be noted that the La/MCM-49 catalyst also showed similar performance attributes with the unmodified MCM-49 catalyst in other parameters. Specifically, as shown in FIG. 4, the methanol conversion was virtually the same between the La/MCM-49 catalyst and the unmodified MCM-49 catalyst at about 100%. As shown in FIG. 5, the toluene conversion of the La/MCM-49 catalyst was found to be approximately 30% which is only slightly below that found for the unmodified MCM-49 catalyst at 31%. Finally, as shown in FIG. 6, total xylene selectivity was found to be approximately 83% for the La/MCM-49 catalyst which was slightly higher than the 81% found for the unmodified MCM-49 catalyst.

### EXAMPLE 6

An La-modified MCM-49 catalyst was made from a mixture of 80 parts (basis: calcined 538° C.) of MCM-49 crystal and 20 parts high surface area Versal™ 300 alumina (basis: calcined 538° C.) and Lanthanum nitrate solution that was combined in a mulling operation. The as-synthesized MCM-49 was mulled first and a mixture of Lanthanum nitrate hexahydrate solution was added gradually to mulled MCM-49 crystals. The remaining water and Versal™ 300 alumina was added, and mulled. The resulting paste was extruded into a 1/20″ Quadra-lobe insert and then dried in a hot pack oven at 121° C. overnight. The dried extrudate was calcined in nitrogen (N₂) at 538° C. to decompose and remove the organic template. The N₂-calcined extrudate was humidified with saturated air and exchanged with 1 N ammonium nitrate to remove sodium. After ammonium nitrate exchange, the extrudate was washed with deionized water to remove residual nitrate ions prior to drying. The ammonium-exchanged extrudate was dried at 121° C. overnight and calcined in air at 538° C. The resulting extrudate had an La content of is 1.96 wt %. The H-formed La-modified extrudate had an Alpha of 410, a Collidine adsorption of 90.7 umoles/g, and a total surface area of 496 m²/g.

### EXAMPLE 7

An La-modified MCM-49 catalyst was made from a mixture of 80 parts (basis: calcined 538° C.) of MCM-49 crystal and 20 parts high surface area Versal-300 alumina (basis: calcined 538° C.) and Lanthanum nitrate solution that was combined in a mulling operation. The as-synthesized MCM-49 crystals was mulled first and alumina binder was added to mulled MCM-49 crystals and completed more mulling step. The desired amount of water and Lanthanum Nitrate Hexahydrate solution was then added gradually and mulled again. The resulting paste was extruded into a 1/20″ Quadra-lobes insert and then dried in a hot pack oven at 121° C. overnight. The dried extrudate was calcined in nitrogen (N₂) at 538° C. to decompose and remove the organic template. The N₂-calcined extrudate was humidified with saturated air and exchanged with 1 N ammonium nitrate to remove sodium. After ammonium nitrate exchange, the extrudate was washed with deionized water to remove residual nitrate ions prior to drying. The ammonium-exchanged extrudate was dried at 121° C. overnight and calcined in air at 538° C. The resulting extrudate had an La content of is 2.0 wt %. The H-formed La-modified extrudate had an Alpha of 400, a Collidine adsorption of 104.2 umoles/g, and a total surface area of 494 m²/g.

While various embodiments have been disclosed herein, modifications thereof can be made without departing from the scope or teachings herein. In particular, many variations and modifications of the systems, apparatus, and processes described herein are possible and are within the scope of the disclosed subject matter. Accordingly, embodiments disclosed herein are exemplary only and are not limiting. As a result, the scope of protection is not limited to the embodiments described herein, but is only limited by the claims that follow, the scope of which shall include all equivalents of the subject matter of the claims. Unless expressly stated otherwise, the steps in a method claim may be performed in any order. The use of identifiers such as (a), (b), (c) before steps in a method claim is not intended to and does not specify a particular order to the steps. Rather the use of such identifiers are used to simplify subsequent reference to such steps. Finally, the use of the term “including” in both the description and the claims is used in an open ended fashion, and should be interpreted as meaning “including, but not limited to.”

1. A process for producing paraxylene, the process comprising:
(a) contacting an aromatic hydrocarbon feed comprising benzene and/or toluene with an alkylating reagent comprising methanol and/or dimethyl ether in at least one alkylation reaction zone in the presence of an alkylation catalyst comprising a molecular sieve having a Constrain Index less than 5 and under alkylation conditions, wherein the alkylation catalyst comprises at least one of a rare earth metal or alkaline earth metal and a binder, and wherein a majority of the at least one rare earth metal or alkaline earth metal is deposited on the molecular sieve; and
(b) producing an alkylated aromatic product comprising xylenes.

2. The process of claim 1, wherein at least 70% of the at least one rare earth metal or alkaline earth metal is deposited on the molecular sieve.

3. The process of claim 2, wherein at least 90% of the at least one rare earth metal or alkaline earth metal is deposited on the molecular sieve.

4. The process of claim 1, wherein the at least one rare earth metal or alkaline earth metal comprises at least one of lanthanum or strontium.

5. The process of claim 4, wherein the at least one rare earth metal or alkaline earth metal comprises lanthanum.

6. The process of claim 1, wherein the alkylation catalyst comprises between about 1 and about 5 wt % of the at least one rare earth metal or alkaline earth metal based on the weight of the final catalyst.

7. The process of claim 1, wherein the molecular sieve has an MWW framework structure.

8. The process of claim 7, wherein the molecular sieve is selected from the group consisting of MCM-22, PSH-3, SSZ-25, ERB-1, ITQ-1, ITQ-2, MCM-36, MCM-49, MCM-56, EMM-10, EMM-12, EMM-13, UZM-8, UZM-8HS, UZM-37, MIT-1, and mixtures thereof.

9. The process of claim 8, wherein the molecular sieve comprises MCM-49.

10. The process of claim 8, wherein the molecular sieve comprises MCM-22.

11. The process of claim 1, wherein the alkylation conditions comprise a temperature between about 500° C. and about 700° C., a pressure of at least 700 kPa-a, and a weight hourly space velocity (WHSV) based on the aromatic hydrocarbon feed and the alkylating reagent from about 10 to 1 hr⁻¹.

12. A process for producing paraxylene, the process comprising:
(a) contacting an aromatic hydrocarbon feed comprising benzene and/or toluene with an alkylating reagent comprising methanol and/or dimethyl ether in at least one alkylation reaction zone in the presence of an alkylation catalyst comprising a molecular sieve of the MWW framework structure under alkylation conditions, wherein the alkylation catalyst comprises lanthanum and a binder, and wherein a majority of the lanthanum is deposited on the molecular sieve; and
(b) producing an alkylated aromatic product comprising xylenes.

13. The process of claim 12, wherein the lanthanum is deposited on the molecular sieve with a mulling operation.

14. The process of claim 13, wherein at least 70% of the lanthanum is deposited on the molecular sieve.

15. The process of claim 14, wherein at least 90% of the lanthanum is deposited on the molecular sieve.

16. The process of claim 12, wherein the alkylation catalyst comprises between about 1 and about 5 wt % of lanthanum based on the weight of the final catalyst.

17. The process of claim 16, wherein the molecular sieve comprises MCM-49.

18. The process of claim 16, wherein the molecular sieve comprises MCM-22.

19. The process of claim 12, wherein the alkylated aromatic product comprises at least 35 wt % of paraxylene, based on the total amount of xylenes.

20. The process of claim 19, wherein the alkylated aromatic product comprises at least 80 xylenes.
